(19) United States
(12) Patent Application Publication
(10) Pub. No.: US 2024/0082426 A1
(43) Pub. Date: Mar. 14, 2024

(54) IONICALLY BONDED COMPOUND OF POLYETHYLENIMINE-CHOLIC ACID WITH GENE TRANSFER ACTIVITY AND USE THEREOF

(71) Applicant: RESEARCH COOPERATION FOUNDATION OF YEUNGNAM UNIVERSITY, Gyeongsan-si, Gyeongsangbuk-do (KR)

(72) Inventors: Kyung-Oh DOH, Daegu (KR); Jae-Won PARK, Daegu (KR)

(73) Assignee: RESEARCH COOPERATION FOUNDATION OF YEUNGNAM UNIVERSITY, Gyeongsan-si, Gyeongsangbuk-do (KR)

(21) Appl. No.: 18/271,676

(22) PCT Filed: Nov. 18, 2021
(86) PCT No.: PCT/KR2021/016986

§ 371 Date: Jul. 11, 2023

(30) Foreign Application Priority Data
Jan. 13, 2021 (KR) 10-2021-0004583

(51) Int. Cl. A61K 48/00; A61K 47/54; A61K 47/59

(52) U.S. Cl. CPC A61K 48/0041; A61K 47/554; A61K 47/59

(57) ABSTRACT

The present invention related to an ionically bonded compound of polyethylene-cholic acid for gene transfer and a use thereof and, more specifically, to a compound in which polyethylenimine and cholic acid are ionically bonded to each other, a preparation method therefor, a gene transfer method thereof, and a use thereof for gene transfer.

### TECHNICAL FIELD

The present disclosure relates to an ionic compound of polyethylenimine-cholic acid having gene transferability and a use thereof, and particularly, to a compound in which polyethylenimine and cholic acid are ionically bonded, a preparation thereof, a gene transfer method thereof, and a use for gene transfer.

### BACKGROUND ART

Gene therapy refers to a method of correcting genetic defects by injecting genetic materials such as pDNA and siRNA into cells of patients or preventing or treating all genetic defects such as cancer, infectious diseases, and autoimmune diseases caused by genetic modification of cells. Such gene therapy is attracting attention as a breakthrough treatment method that enables cancer treatment or treatment of diseases caused by genetic modification.

For such gene therapy, research is actively being conducted on the development of gene deliveries such as viruses, liposomes, and polymers capable of intracellularly delivering genetic materials having therapeutic effects.

On the other hand, biocompatible polymers are constituents of the drug delivery to be used for development of effective systems to deliver various therapeutics such as chemical drugs, contrast agents, peptides, proteins, and genetic materials.

Thereamong, polyethylenimine (PEI), a cationic polymer, is composed of a high concentration of cationic amine groups, with a capability of forming colloidal particles by compressing negatively charged nucleic acid substances and an ability to enter the cell through endocytosis.

Gene transfer is divided into three main categories such as passage through the cell membrane, endosomal escape, and passage through the nuclear membrane, and a complex using polyethylenimine may effectively perform endosomal escape through a proton sponge effect using pH buffering ability, unlike other gene deliveries. However, polyethylenimine which is relatively polymeric is able to effectively transfer genes but has strong cytotoxicity, and low-molecular-weight polyethylenimine has low cytotoxicity but relatively poor gene transfer efficiency.

On the other hand, cholic acid exhibits high hydrophilicity and biocompatibility compared to cholesterol and may efficiently destabilize cell membranes due to amphiphilicity, such that it is effective in constructing the gene delivery. In addition, as a ligand for steroid receptors expressed on the nuclear membrane, it may improve gene transfer efficiency. Previous studies using cholic acid to synthesize polyethylenimine derivatives have enhanced gene transfer efficiency, but there are difficulties in synthesizing derivatives through complicated chemical formulas.

Based on this, the present disclosure simply enables formation of a compound as a derivative, in which various kinds of cholic acid and polyethylenimine having various molecular weights are ionically bonded, revealing the utility of a gene delivery thereof.

### DISCLOSURE OF THE INVENTION

### Technical Goals

An object of the present disclosure is to provide a gene delivery with low toxicity and effective gene transfer efficiency, a preparation method thereof, and an intracellular gene transfer method using the same.

### Technical Solutions

In order to achieve the above object, the present disclosure provides a gene delivery in which polyethylenimine and cholic acid are ionically bonded and which is represented by the following Chemical Formula 1.

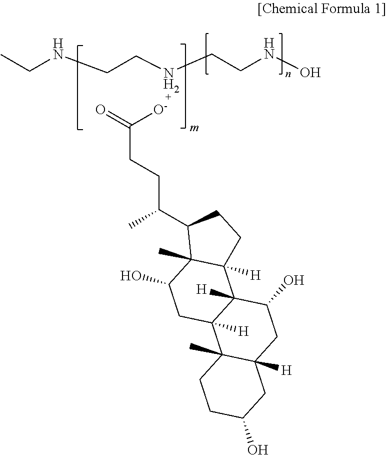

In the Chemical Formula 1, m is an integer of 2 to 930, and n is an integer of 58 to 930.

In addition, the present disclosure provides a composition for gene transfer, including the gene delivery and a gene.

In addition, the present disclosure provides a preparation method of a gene delivery, including (a) dissolving polyethylenimine in an alcohol solution and adding an acid solution to carry out reaction; and (b) mixing cholic acid with the solution and carrying out reaction followed by sonicating to obtain the gene delivery.

In addition, the present disclosure provides a method of transferring a gene, including bringing the gene delivery into contact with cells.

### Advantageous Effects

A derivative of polyethylenimine-cholic acid according to the present disclosure has low toxicity and excellent gene transfer efficiency, such that it is useful for gene transfer to be widely applicable to gene therapy.

### BRIEF DESCRIPTION OF DRAWINGS

FIG. 1 shows a diagram illustrating a process of preparing a compound in which polyethylenimine and cholic acid are ionically bonded using three types of polyethylenimine and three types of cholic acid.

FIG. 2 shows results of analyzing lithocholated linear polyethylenimine (LPL) via Fourier transform infrared spectroscopy (FT-IR).

FIG. 3 shows results of comparing the gene transfer efficiency and cytotoxicity of lithocholated linear polyethylenimine (LPL) ionically bonded with PLC, using covalent bonds in Chinese hamster ovarian (CHO) cells and cervical cancer cells (HeLa).

FIG. 4 shows results of evaluating transfection efficiency and cytotoxicity of a gene delivery synthesized in Chinese hamster ovarian (CHO) cells.

FIG. 5 shows results of evaluating transfection efficiency and cytotoxicity of a gene delivery synthesized in Chinese hamster ovarian (CHO) cells depending on a dosage (weight ratio) of polyethylenimine and DNA.

FIG. 6 shows results of evaluating transfection efficiency of a compound according to the present disclosure in accordance with the pH control.

FIG. 7 shows results of evaluating gene transfer efficiency and cytotoxicity of a gene delivery prepared by adjusting the optimal ratio of DNA to compound (1:4) and pH (6.9 to 7.1).

### BEST MODE FOR CARRYING OUT THE INVENTION

The present disclosure has been completed by identifying that a compound in which polyethylenimine and cholic acid are ionically bonded has low toxicity and excellent gene transfer efficiency in various cell lines (Chinese hamster ovarian (CHO) cells, cervical cancer cells (HeLa).

Thus, the present disclosure provides a gene delivery in which polyethylenimine and cholic acid are ionically bonded and which is represented by the following Chemical Formula 1.

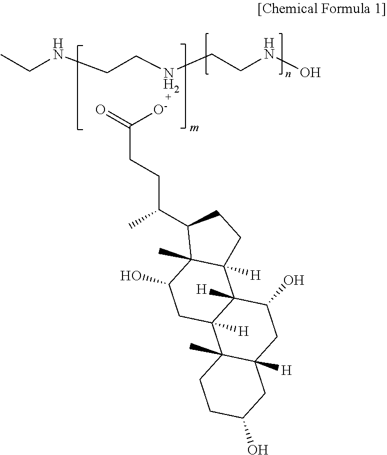

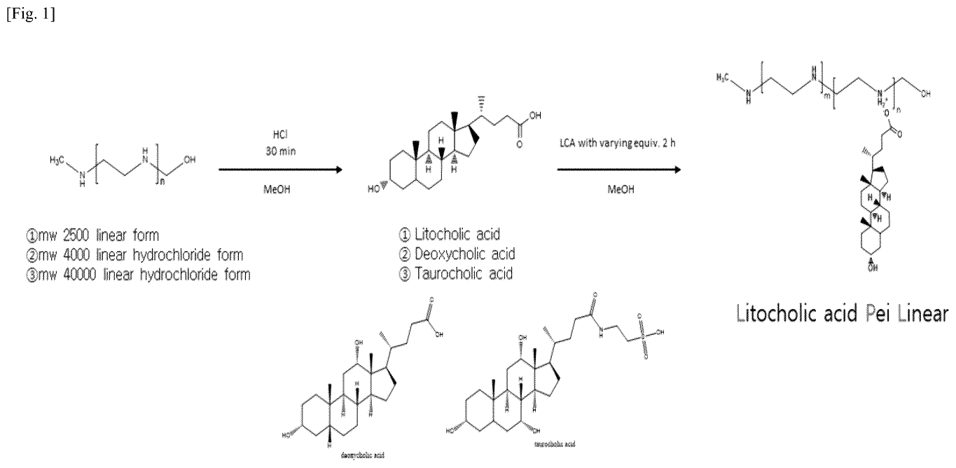

In the Chemical Formula 1, m is an integer of 2 to 930, and n is an integer of 58 to 930. FIG. 1 shows a diagram illustrating a process in which polyethylenimine and cholic acid are ionically bonded.

The polyethylenimine may be linear polyethylenimine (linear PEI) or branched polyethylenimine (branched PEI). Preferably, it may be linear polyethylenimine.

In the implementation of the present disclosure, gene transfer efficiency of the linear polyethylenimine may decrease when the molecular weight is small, and cytotoxicity may appear when the molecular weight is large. It is known that the number of the branch chain in branched polyethylenimine is about one per every 3 to 3.5 nitrogen atoms in the main chain, and such polyethylenimine is soluble in water, alcohol, glycol, dimethylformamide, tetrahydrofuran, and esters, while it is known to be insoluble in high-molecular-weight hydrocarbons, oleic acid, and diethyl ether. In addition, polyethylenimine may slowly react with most chlorinated solvents to be cross-linked with ketones.

A weight-average molecular weight of polyethylenimine may be 2,500 to 40,000. If the weight-average molecular weight is less than 2,500, there is a limitation in transfection and also in cytotoxicity if it is more than 40,000, such that it is desirable to use those within the above range.

The cholic acid may be one or more types selected from the group consisting of lithocholic acid, deoxycholic acid, and taurocholic acid, but is not limited thereto.

In the present disclosure, the compound was named according to the type of cholic acid and the type of polyethylenimine. For example, a compound in which lithocholic acid and linear polyethylenimine (PEI Linear) are used is called lithocholic acid PEI linear (LPL).

The gene may be selected from the group consisting of gDNA, cDNA, plasmid DNA, mRNA, tRNA, rRNA, antisense nucleotide, missense nucleotide, and protein-producing nucleotide. For example, the gene may be a gene expressing an epidermal growth factor (EGF), fibroblast growth factor (FGF), platelet-derived growth factor (PDGF), transforming growth factor-b (TGF-b), vascular endothelial growth factor (vEGF) or insulin, but is not necessarily limited thereto.

In addition, the present disclosure provides a composition for gene transfer, including the gene delivery; and a gene.

In this case, it is preferable to include the gene delivery and gene in a weight ratio of 4 to 6:1 since it shows low toxicity and the most efficient gene transfer.

The composition of the present disclosure includes lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil that are commonly used as a pharmaceutically acceptable carrier, but is not limited thereto. The composition of the present disclosure may further include lubricants, wetting agents, sweetening agents, flavoring agents, emulsifying agents, suspending agents, and preservatives, in addition to the above components.

In addition, the present disclosure provides a method of preparing a gene delivery, including (a) dissolving polyethylenimine in an alcohol solution and adding an acid solution to carry out reaction; and (b) mixing cholic acid with the solution and carrying out reaction followed by sonicating to obtain the gene delivery represented by the Chemical Formula 1.

To prepare the compound in which polyethylenimine and cholic acid are ionically bonded, in the step (a), polyethylenimine is dissolved in the alcohol solution and the acid solution is added to carry out reaction. The alcohol solution is one or more types selected from the group consisting of methanol, ethanol, propanol, butanol, pentanol and hexanol, but is not necessarily limited thereto.

The step (b) is a step of obtaining the gene delivery represented by the Chemical Formula 1 by mixing cholic acid with the solution and carrying out reaction followed by sonicating, wherein cholic acid is mixed separately in the alcohol solution to be added to the solution obtained in the step (a).

The solution obtained in the step (b) is subjected to vacuum condensation to completely remove a solvent and then sonicated to obtain the compound in which polyethylenimine and cholic acid are ionically bonded.

In this case, it is preferable to adjust the pH of the solution including polyethylenimine and cholic acid obtained in the step (b) to 6.9 to 7.1. This is because the ionic compound of polyethylenimine and cholic acid may be prepared most efficiently.

Further, it is most preferable to carry out reaction for 1 to 3 hours in terms of yield after mixing cholic acid in the step (b), which may be changed according to the reaction conditions.

In addition, the present disclosure provides a method of transferring a gene, including bringing the gene delivery represented by the Chemical Formula 1 into contact with cells in vitro or in vivo.

### MODES FOR CARRYING OUT THE INVENTION

Hereinafter, the present disclosure will be described in detail according to an example embodiment that does not limit the present disclosure. It is apparent that the following example embodiments of the present disclosure are only for the purpose of embodying the present disclosure, but do not restrict or limit the scope of rights of the present disclosure. Therefore, what may be easily inferred from the detailed description and example embodiments of the present disclosure by an expert in the art to which the present disclosure pertains is interpreted as falling within the scope of the right of the present disclosure.

### <Example 1> Synthesis of Ionically Bonded Gene Delivery

The present disclosure will be described in more detail in the following example embodiments. These example embodiments are for illustration only, and the scope of the present disclosure is not limited by these example embodiments.

1-1. Synthesis of Lithocholated Linear Polyethylenimine (LPL)

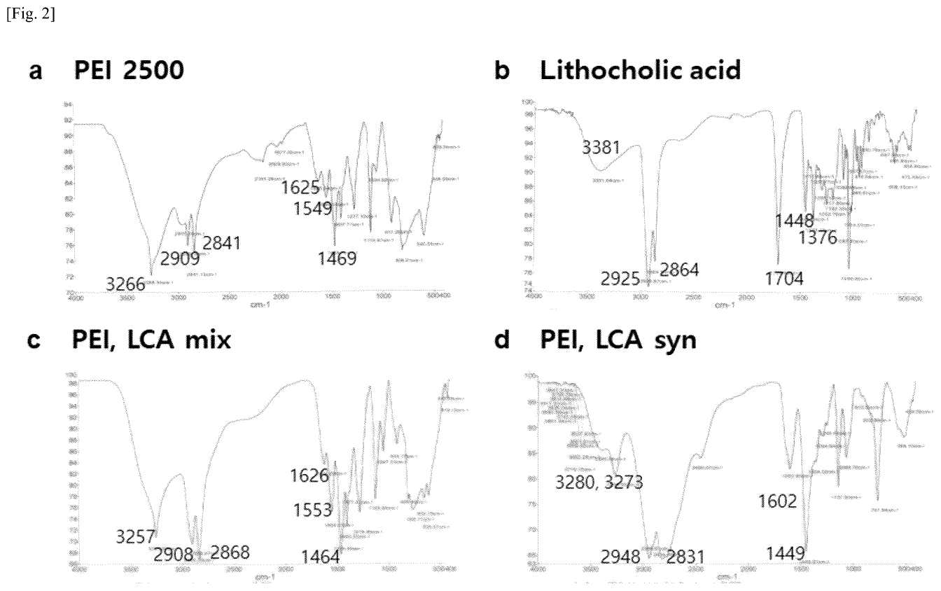

Linear polyethylenimine (weight-average molecular weight of 2,500) was dissolved in methanol, an aqueous hydrochloric acid (HCl) solution was added, and reaction was carried out at room temperature for 30 minutes. Lithocholic acid dissolved in methanol was added, and the reaction was carried out again for 2 hours. After the end of the reaction, a lipid film formulation was formed by a rotary evaporation concentrator. The solvent was completely removed through vacuum condensation. Thereafter, distilled water was added, and the gene delivery was formed through ultrasound treatment (FIG. 1). In addition, completion of the synthesis was checked using Fourier transform infrared spectroscopy (FT-IR) (FIG. 2).

1-2. Synthesis of Deoxycholated Linear Polyethylenimine (DPL)

Preparation was performed in the same manner as in Example 1-1 above, using deoxycholic acid instead of lithocholic acid (FIG. 1).

1-3. Synthesis of Taurocolated Linear Polyethylenimine (TPL)

Preparation was performed in the same manner as in Example 1-1 above, using taurocholic acid instead of lithocholic acid (FIG. 1).

1-4. Synthesis of Lithocholated Linear Polyethylenimine (LPH)

Preparation was performed in the same manner as in Example 1-1 above, using linear polyethylenimine with the weight-average molecular weight of 4,000 instead of that with the weight-average molecular weight of 2,500 (FIG. 1).

1-5. Synthesis of Deoxycholated Linear Polyethylenimine (DPH)

Preparation was performed in the same manner as in Example 1-2 above, using linear polyethylenimine with the weight-average molecular weight of 4,500 instead of that with the weight-average molecular weight of 2,500 as well as deoxycholic acid instead of lithocholic acid (FIG. 1).

1-6. Synthesis of Taurocolated Linear Polyethylenimine (TPH)

Preparation was performed in the same manner as in Example 1-3 above, using linear polyethylenimine with the molecular weight of 4,000 instead of that with the weight-average molecular weight of 2,500 as well as taurocholic acid instead of lithocholic acid (FIG. 1).

1-7. Synthesis of Lithocholated Linear Polyethylenimine (LPM)

Preparation was performed in the same manner as in Example 1-1 above, using linear polyethylenimine with the weight-average molecular weight of 40,000 instead of that with the weight-average molecular weight of 2,500 (FIG. 1).

1-8. Synthesis of Deoxycholated Linear Polyethylenimine (DPM)

Preparation was performed in the same manner as in Example 1-2 above, using linear polyethylenimine with the weight-average molecular weight of 40,000 instead of that with the weight-average molecular weight of 2,500 as well as deoxycholic acid instead of lithocholic acid (FIG. 1).

1-9. Synthesis of Taurocolated Linear Polyethylenimine (TPM)

Preparation was performed in the same manner as in Example 1-3 above, using linear polyethylenimine with the weight-average molecular weight of 40,000 instead of that with the weight-average molecular weight of 2,500 as well as taurocholic acid instead of lithocholic acid (FIG. 1).

### <Comparative Example 1> Synthesis of Covalently Bonded Gene Delivery (PLC)

Prepared were gene deliveries covalently bonded by a known amidization method under a 1,1′-carbonyldiimidazole (CDI) catalyst (Biomaterials, 217 (2019), p. 119296).

### <Experimental Example 1> Evaluation on Cytotoxicity in Chinese Hamster Ovarian (CHO) Cells and Cervical Cancer Cells (HeLa)

The present inventors performed transfection in Chinese hamster ovarian (CHO) cells and cervical cancer cells (HeLa) for the compound prepared in Example 1 above to evaluate cytotoxicity. Specifically, the CHO cell lines (KCLB, Republic of Korea) were cultured in a medium including F-12K (Hyclone, USA), 10% bovine serum (FBS, Hyclone), 1% penicillin/streptomycin (Hyclone), and 1% L-glutamine. Cells with passage number 5-7 were used in the study. After culturing 8,000 CHO cells per well on a 96-well plate for a day, a transfection experiment was performed when more than 70% of the cells in each well were grown.

Next, the HeLa cell lines (KCBL, Republic of Korea) were cultured in a culture medium including MEM (Hyclone, USA), 10% bovine serum (FBS, Hyclone), 1% penicillin/streptomycin (Hyclone), and 1% L-glutamine. Cells with passage number 5-7 were used in the study. After culturing 10,000 HeLa cells per well on a 96-well plate for a day, a transfection experiment was performed when more than 70% of the cells in each well were grown.

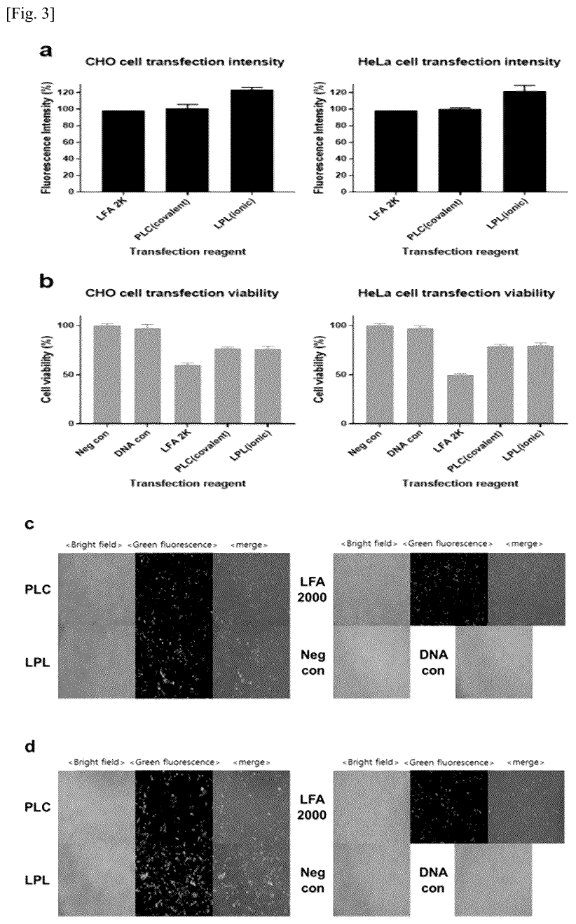

Each well was replaced with 150 μl of bovine serum-containing medium, and a plasmid DNA-lipid (Example 1) mixture solution was prepared. To check transfection, green fluorescence (GFP) inserted plasmid DNA was used as the plasmid DNA, and 1 μg of plasmid DNA was mixed with 10 μl of bovine serum-free medium for preparation. PLC synthesized using covalent bonds and the compound (LPL) of Example 1-1 synthesized using ionic bonds were mixed, by 4 μg of each, with 10 μl of bovine serum-free medium, respectively. The two dilutions were mixed well and left at room temperature for 30 minutes, and the mixture solution prepared thereby was added to a plate, followed by culture in a CO₂ incubator at 37° C. for 24 hours. The expressed green fluorescent protein was observed under fluorescence microscopy, and cytotoxicity was evaluated via WST assay (FIG. 3).

FIG. 3-a shows a result of measuring the expression level of fluorescence in two cell lines by a fluorometer, and in the case of PLC using covalent bonds, the expression was similar compared to LFA2000, while in the case of LPL using ionic bonds, the transfer efficiency was increased by more than 20%. Therefore, it was grasped that the delivery using ionic bonds better transfers nucleic acid substances into the cell than that using covalent bonds.

FIG. 3-b shows a result of conducting a cytotoxicity experiment in two cell lines, in which LFA2K showed significantly greater cytotoxicity compared to an untreated group, while the two synthesized gene deliveries showed significantly reduced cytotoxicity.

As a result of microscopic observation of cell viability and fluorescence expression in FIGS. 3-c and 3-d, it is possible to check a significantly higher cell viability compared to a control group in the bright field and also observe significantly increased green fluorescence in the expression of green fluorescence. Thereby, it was found that the gene delivery (LPL) using ionic bonds is a gene delivery substance with reduced cytotoxicity compared to commercially available products, with more increased transferability than the gene delivery (PLC) using covalent bonds.

### <Experimental Example 2> Evaluation on Cytotoxicity in Chinese Hamster Ovarian (CHO) Cells

CHO cell lines (KCLB, Republic of Korea) were cultured in culture media including F-12K (Hyclone, USA)+10% bovine serum (FBS, Hyclone), 1% penicillin/streptomycin (Hyclone), and 1% L-glutamine, and cells with passage number 5-7 were used in the study. After culturing 8,000 CHO cells on a 96-well plate for a day, a transfection experiment was performed when more than 70% of the cells in each well were grown

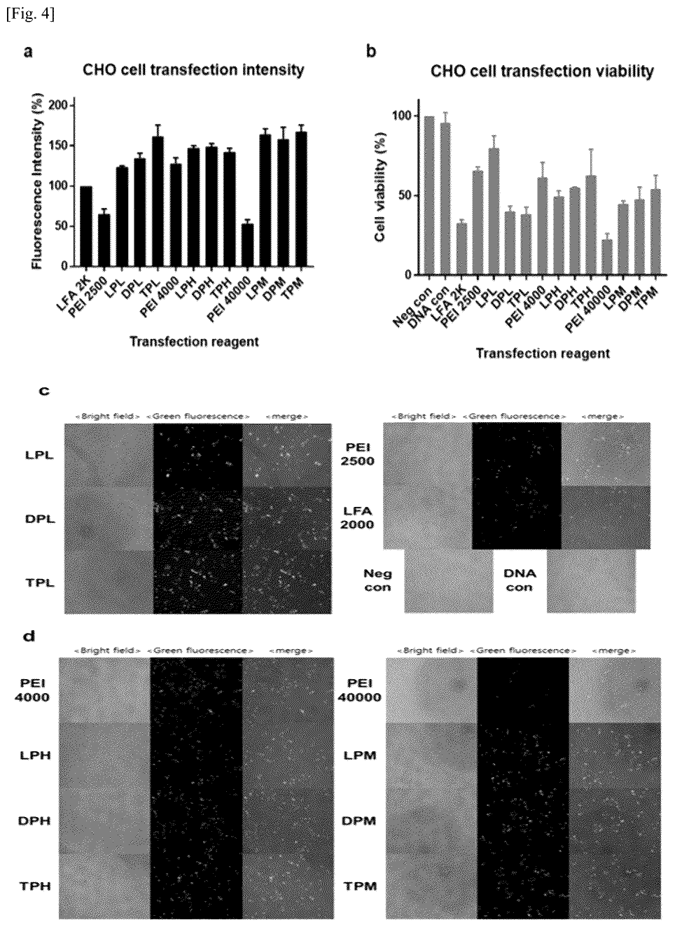

Each well was replaced with 150 μl of bovine serum-containing medium, and a plasmid DNA-lipid (Examples 1-1 to 1-9) mixture solution was prepared. To check transfection, green fluorescence (GFP) inserted plasmid DNA was used as plasmid DNA, and 1 μg of plasmid DNA was mixed with 10 μl of bovine serum-free medium for preparation. 4 μg of compounds in Examples 1-1 to 1-9 were mixed in 10 μl of bovine serum-free medium respectively for preparation. The two dilutions were thoroughly mixed and left at room temperature for 30 minutes, and the mixture solution prepared thereby was added to the plate, followed by culture in a CO₂ incubator at 37° C. for 24 hours. The expressed green fluorescent protein was observed under fluorescence microscopy, and cytotoxicity was evaluated via WST assay (FIG. 4).

FIG. 4-a shows a result of measuring the expression level of fluorescence by a fluorometer, in which, when polyethylenimine (2500, 40000) was solely treated, only a half amount of expression was observed compared to LFA2000, while most of the synthesized gene deliveries (Examples 1-1 to 1-9) increased significantly. Therefore, it was found that the synthesized gene deliveries have an ability to transfer nucleic acid substances into cells with desirable efficiency.

FIG. 4-b shows a result of conducting a cytotoxicity experiment, in which LFA2K showed very significant cytotoxicity than the untreated group, while the synthesized gene deliveries showed reduced cytotoxicity than LFA2K. Therefore, it was found that the synthesized gene deliveries were those with low cytotoxicity.

FIGS. 4-c and 4-d show results of microscopic observation of cell viability and fluorescence expression, in which a significantly higher cell viability may be observed compared to LFA2K in a bright field, and a significantly increased green fluorescence may be observed in the expression of green fluorescence. Thereby, it was found that the synthesized gene deliveries are advanced gene delivery substances with increased nucleic acid transferability while reducing cytotoxicity compared to commercially available products.

### <Experimental Example 3> Evaluation on Transfection Efficiency According to a Ratio of DNA and Synthetic Compounds

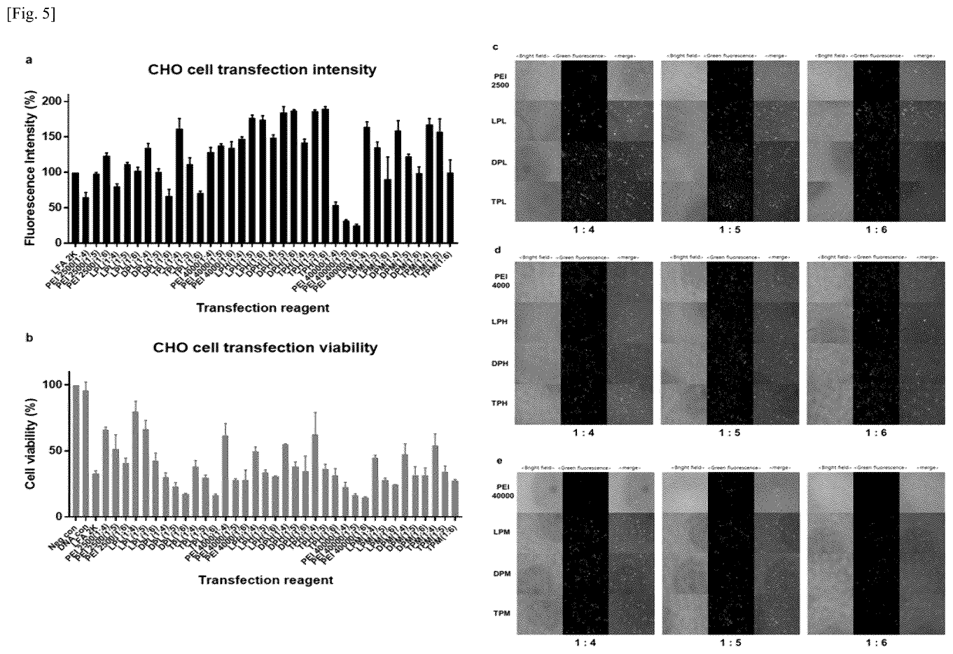

In order to identify the difference according to the DNA and compound ratio (weight ratio), DNA and compounds in Examples 1-1 to 1-9 were used in a ratio of 1:4, 1:5, and 1:6 to determine the transformation efficiency according to the DNA and compound ratio (FIG. 5). The experimental method is the same as in Experimental Examples 1 and 2 above.

FIG. 5-a shows a result of measuring the gene delivery efficiency according to the DNA:compound ratio by the expression level of fluorescence, in which, in most of the results, the transferability was better than that of Lipofectamine 2000 (LFA 2K), and in a specific ratio, the nucleic acid transferability was further increased than in the results of Experimental Example 2.

FIG. 5-b shows a result of conducting a cytotoxicity experiment, showing that cytotoxicity increased as a proportion of the compound increased, with the best cell viability in the ratio of 1:4 to 1:5. The most optimal ratio was 1:4.

FIGS. 5-c, 5-d, and 5-e show results of microscopic observation of cell viability and fluorescence expression, corresponding to FIGS. 5-a and 5-b.

### <Experimental Example 4> Evaluation on Transfection Efficiency According to pH Adjustment

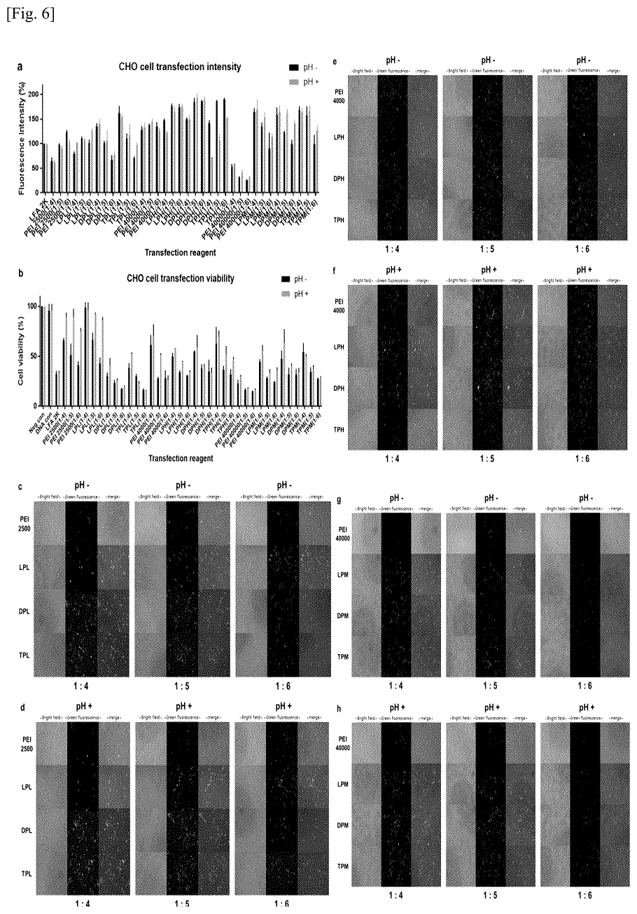

In order to identify the difference in transformation efficiency of the synthesized gene delivery compound according to pH adjustment, transformation efficiency was determined in the same manner as in Experimental Example 3 except for adjustment of the pH to 7.00±0.1 (represented as pH+) when preparing the compounds in Examples 1-1 to 1-9 (FIG. 6).

FIG. 6-a shows a result of measuring the gene transfer efficiency according to pH by the expression level of fluorescence, and it was found that the expression level of fluorescence was not greatly affected by pH. On the other hand, FIG. 6-b shows a result of conducting a cytotoxicity experiment, in which the compound adjusted to pH 6.9 to 7.1 showed a higher cell viability than that without adjustment.

FIGS. 6-c, 6-d, 6-e, 6-f, 6-g, and 6-h show a result of microscopic observation of cell viability and fluorescence expression, corresponding to FIGS. 6-a and 6-b.

### <Experimental Example 5> Gene Transfer Efficiency and Cytotoxicity in Cervical Cancer Cells (HeLa)

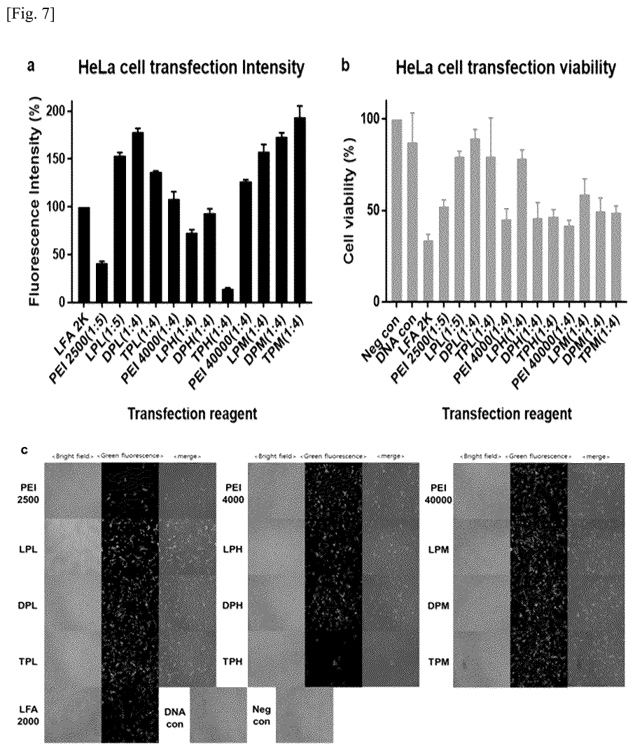

MEM media (Cyclone, USA) were used for HeLa cell lines (KCBL, Republic of Korea), and 10,000 cells were placed per well in the 96-well plate. In Examples 1-1 to 1-9, with adjustment to conditions that showed the best results in Experimental Examples 1, 2, and 3 (DNA:compound ratio=1:4, pH 6.9 to 7.1), all other processes were performed in the same manner as in Experimental Examples 3 and 4 so as to identify gene transfer efficiency and cytotoxicity (FIG. 7).

FIG. 7-a shows a result of measuring the expression level of fluorescence by a fluorometer, in which the synthesized deliveries except TPH among the synthesized gene deliveries showed similar or favorable gene transfer efficiency with Lipofectamine 2000 (LFA2K).

FIG. 7-b shows a result of conducting a cytotoxicity experiment, in which LFA2K showed very significant cytotoxicity compared to the untreated group, while the synthesized gene deliveries showed reduced cytotoxicity.

FIG. 7-c shows a result of microscopic observation of cell viability and fluorescence expression, corresponding to FIGS. 7-a and 7-b.

As described above, the present disclosure enables easy formation of a derivative of a compound in which various types of cholic acid and polyethylenimine having various molecular weights are ionically bonded, revealing the efficacy of a gene delivery thereof. The ionic compound of polyethylenimine-cholic acid according to the present disclosure has low toxicity and excellent gene transfer efficiency, such that it is useful for gene transfer to be widely applicable to gene therapy.

As a specific part of the present disclosure is described in detail, it is apparent to those skilled in the art that such a specific description is only a preferred example embodiment, and the scope of the present disclosure is not limited thereby. Thus, the substantive scope of the present disclosure will be defined by the attached claims and their equivalents.

1. A gene delivery in which polyethylenimine and cholic acid are ionically bonded and which is represented by the following Chemical Formula 1: 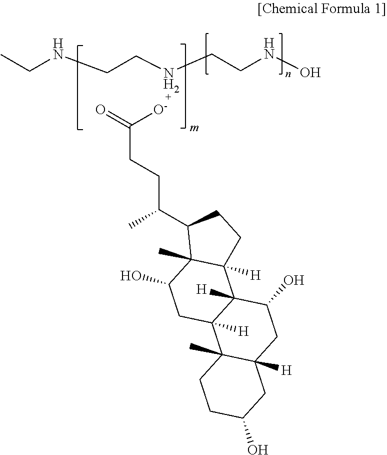
wherein, in the Chemical Formula 1,
m is an integer of 2 to 930, and n is an integer of 58 to 930.

2. The gene delivery of claim 1, wherein the gene is selected from the group consisting of gDNA, cDNA, plasmid DNA, mRNA, tRNA, rRNA, antisense nucleotide, missense nucleotide, and protein-producing nucleotide.

3. The gene delivery of claim 1, wherein the polyethylenimine is a linear or branched polyethylenimine with a weight-average molecular weight of 2,500 to 40,000.

4. The gene delivery of claim 1, wherein the cholic acid is one or more types selected from the group consisting of lithocholic acid, deoxycholic acid, and taurocholic acid.

5. A gene delivery method, comprising:
providing a composition comprising a gene delivery in which polyethylenimine and cholic acid are ionically bonded and which is represented by the following Chemical Formula 1 and a gene, 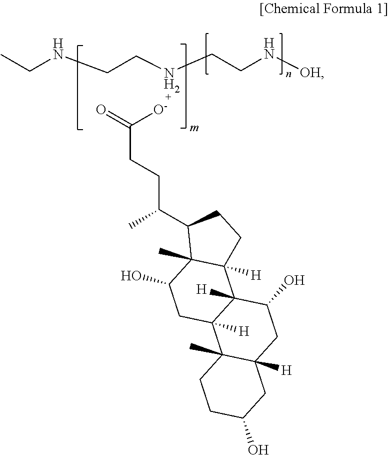
wherein, in the Chemical Formula 1,
m is an integer of 2 to 930, and n is an integer of 58 to 930; and
contacting the composition with cells.

6. The method of claim 5, wherein the gene is selected from the group consisting of gDNA, cDNA, plasmid DNA, mRNA, tRNA, rRNA, antisense nucleotide, missense nucleotide, and protein-producing nucleotide.

7. The method of claim 5, wherein the gene delivery and the gene are included in a weight ratio of 4 to 6:1.

8. A method of preparing a gene delivery, the method comprising:
(a) dissolving polyethylenimine in an alcohol solution and adding an acid solution to carry out reaction; and
(b) mixing cholic acid with the solution and carrying out reaction followed by sonicating to obtain the gene delivery in which the polyethylenimine and the cholic acid are ionically bonded and which is represented by the following Chemical Formula 1, 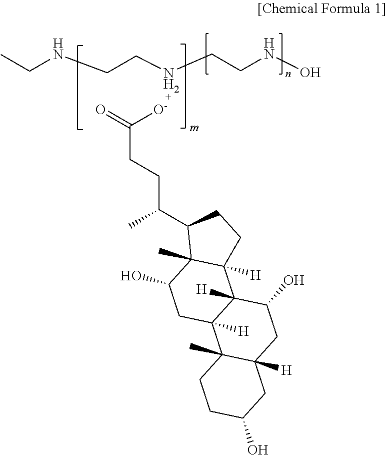
wherein, in the Chemical Formula 1,
m is an integer of 2 to 930, and n is an integer of 58 to 930.

9. The method of claim 8, wherein the step (b) further comprises adjusting the pH of the solution in which polyethylenimine and cholic acid are included to 6.9 to 7.1.

10. (canceled)
